(19) United States
(12) Patent Application Publication
(10) Pub. No.: US 2023/0016631 A1
(43) Pub. Date: Jan. 19, 2023

(54) METHODS FOR COLOR-BLINDNESS REMEDIATION THROUGH IMAGE COLOR CORRECTION

(71) Applicants: VISU, INC., Palo Alto, CA (US); WAVESHIFT LLC, Palo Alto, CA (US)

(72) Inventors: David William Olsen, Fremont, CA (US); Michael Benjamin Selkowe Fertik, Palo Alto, CA (US); Thomas W. Chalberg, Jr., Menlo Park, CA (US)

(21) Appl. No.: 17/783,619

(22) PCT Filed: Dec. 9, 2020
(86) PCT No.: PCT/US2020/064132

§ 371 Date: Jun. 8, 2022

Related U.S. Application Data
(60) Provisional application No. 62/945,849, filed on Dec. 9, 2019.

(51) Int. Cl. G09G 3/20

(52) U.S. Cl. CPC G09G 3/2003; G09G 2340/06; G09G 2320/0666

(57) ABSTRACT

A method includes receiving information about a color blindness of a viewer and an electronic display; receiving information about an input image including input R, G, and B values for pixels composing the input image; transforming each pixel's input values to corresponding x and y values in a CIE color space; identifying which of the pixels are within a filter area in the CIE color space; adjusting a color of the pixels that are within the filter area away from a confusion line for the color blind person to provide a corresponding color-modified pixel; generating a color-modified image composed of the color-modified and original pixels; and displaying the color-modified image with the electronic display.

### CROSS-REFERENCE TO RELATED APPLICATION

This application claims priority to U.S. Provisional Application Ser. No. 62/945,849, filed Dec. 9, 2019, the contents of which are herein incorporated in their entirety.

### FIELD OF THE DISCLOSURE

The disclosure relates to image color correction and more specifically, to image color correction for the remediation of color vision deficiency.

### BACKGROUND

Electronic displays are ubiquitous in today's world. For example, mobile devices such as smartphones and tablet computers commonly use a liquid crystal display (LCD) or an organic light emitting diode (OLED) display. LCDs and OLED displays are both examples of flat panel displays and are also used in desktop monitors, TVs, and automotive and aircraft displays.

Many color displays, including many LCD and OLED displays, spatially synthesize color. In other words, each pixel is composed of three sub-pixels that provide a different color. For instance, each pixel can have a red, green, or blue sub-pixel, or a cyan, magenta, or yellow sub-pixel. The color of the pixel, as perceived by a viewer, depends upon the relative proportion of light from each of the three sub-pixels.

Color gamuts for modern device displays utilize a portion of the colors detectable by a typical human eye to reproduce images for display to a user. A useful representation of a display's color gamut can be made using a CIE color space, which represents the visible color spectrum in a two-dimensional coordinate space. For full color displays using three sub-pixel colors, the color gamut is usually represented by a triangle in the CIE color space where the vertices correspond to the x-y coordinates of each sub-pixel. Typically, higher end displays offering larger gamuts will utilize a larger area in the CIE color space. Example color gamuts include sRGB, Adobe RGB, and NTSC.

Color vision deficiency (e.g., color blindness) is the decreased user ability to perceive certain colors or differences in colors and corresponds to lower cone cell stimulation for a portion of the CIE color space. Daily tasks can be impaired such as selecting ripe fruit, choosing clothing, and reading traffic lights. Additionally, users experiencing color blindness cannot perceive a portion of displayed gamut when viewing images or videos on a device using a modern display gamut. Colorblind users thereby lose image detail and color specificity in color-deficient perception regions of the display gamut.

### SUMMARY

This disclosure features technology for modifying the color of individual pixels in a displayed image to reduce color confusion for users experiencing color blindness. The technology involves defining color filtration regions within a CIE color space corresponding to the display color gamut. Regions can be defined by software or hardware and can be passively or actively defined. Regions corresponding to red-green (e.g., protanopia or deuteranopia), or blue-yellow (e.g., tritanopia) color blindness can be defined.

An image including a matrix of pixels is received and the color information of each image pixel is transformed from into the two-dimensional CIE color space coordinates, e.g., from the original RGB values or other higher dimensional native format.

Pixels falling within the filtration region based on transformed x-y coordinate locations are flagged for modification to a color that corresponds to a CIE region of lower color confusion. Pixels with coordinates falling outside the filtration region can be left unflagged. The color of flagged pixels is modified (e.g., translated) from their respective original CIE coordinates to a new set of CIE coordinates to reduce color confusion.

The shifting of flagged pixel x-y colors can be performed as a transformation of the pixel's x-y coordinates within the CIE color space before transforming the coordinates back to the higher dimensional space (e.g., RGB values) for display to the user.

Alternatively, the flagged pixels color can be shifted by directly transforming respective subpixel intensities in a manner that remediates associated color confusion. For example, protanopia remediation can correspond to a reduction of red sub-pixel intensities and a corresponding increase in blue sub-pixel intensities.

The color-shifted flagged pixels are then recombined with unflagged pixels (which retain their original colors) to form a color-shifted version of the original image, and the combined image is displayed to the user. The color-shifted image can facilitate an improved viewing experience for a color blind user.

In general, in a first aspect, the invention features a method including receiving information about a color blindness of a viewer of an electronic display; receiving information about an input image including input R, G, and B values for each of a plurality of pixels composing the input image; transforming each pixel's input R, G, and B values to a corresponding x value and a corresponding y value, the x and y values being in a CIE color space; identifying which of the pixels are within a filter area in the CIE color space based on the x, y values; adjusting a color of each of the pixels that are within the filter area away from a confusion line in the CIE space for the color blind person to provide a corresponding color-modified pixel; generating a color-modified image composed of the color-modified pixels and original pixels, the original pixels corresponding to pixels having the input R, G, and B values located outside the filter area in the CIE color space; and displaying the color-modified image with the electronic display.

Embodiments may include one or more of the following features. The method can include defining the filter area in the CIE color space based on the information about the color blindness of the viewer. The filter area can be defined by specifying one or more lines defining a border of the filter area. The one or more lines define a polygon in the CIE color space within a color gamut of the electronic display. The color of each pixel within the filter area can be adjusted by translating the x, y coordinates of the pixel in the CIE color space according to a geometric formula.

The electronic display has a color gamut defined by a triangle in the CIE color space and adjusting the color of each pixel that is within the filter area away from the line of color confusion includes translating the x, y coordinates of the pixel along a line parallel to a side of the triangle defining the color gamut. The x, y coordinates for a pixel within the filter area can be translated by a fractional amount of a distance of the x, y coordinates to a side of the triangle defining the color gamut.

In some embodiments, the confusion line can be for a protanope and the side of the triangle defining the color gamut can be the side defined by blue and red sub-pixels of the electronic display. The confusion line can be for a deuteranope and the side of the triangle defining the color gamut can be the side defined by blue and green sub-pixels of the electronic display. The confusion line can be for a tritanope and the side of the triangle defining the color gamut can be the side defined by blue and red sub-pixels of the electronic display.

In some embodiments, the x, y values in the CIE color space for one or more of the color-modified pixels lies within the filter area. The x, y values in the CIE color space for one or more of the color-modified pixels lies outside the filter area. The color of each pixel within the filter area can be adjusted by redistributing relative weights of the input R, G, B values for each pixel within the filter area.

The confusion line can be for a protanope and redistributing relative weights of the input R, G, B values for each pixel within the filter area includes increasing a ratio of the R value to the B value. The confusion line can be for a dueteranope and redistributing relative weights of the input R, G, B values for each pixel within the filter area includes increasing a ratio of the B value to the G value. The confusion line can be for a tritanope and redistributing relative weights of the input R, G, B values for each pixel within the filter area includes increasing a ratio of the R value to the B value.

In a second aspect, the invention features a computer readable medium containing program instructions for displaying a color-modified image with an electronic display, wherein execution of the program instructions by one or more processors of a computer system causes the one or more processors to carry out the steps of receiving information about a color blindness of a viewer of an electronic display; receiving information about an input image including input R, G, and B values for each of a plurality of pixels composing the input image; transforming each pixel's input R, G, and B values to a corresponding x value and a corresponding y value, the x and y values being in a CIE color space; identifying which of the pixels are within a filter area in the CIE color spaced based on the x, y values; adjusting a color of each of the pixels that are within the filter area away from a confusion line in the CIE space for the color blind person to provide a corresponding color-modified pixel; generating a color-modified image composed of the color-modified pixels and original pixels, the original pixels corresponding to pixels having the input R, G, and B values located outside the filter area in the CIE color space; and displaying the color-modified image with the electronic display.

In a third aspect, the invention features a system including an electronic display; and a data processing apparatus programmed to: (a) receive information about a color blindness of a viewer of the electronic display; (b) receive information about an input image including input R, G, and B values for each of a plurality of pixels composing the input image; (c) transform each pixel's input R, G, and B values to a corresponding x value and a corresponding y value, the x and y values being in a CIE color space; (d) identify which of the pixels are within a filter area in the CIE color spaced based on the x, y values; (e) adjust a color of each of the pixels that are within the filter area away from a confusion line in the CIE space for the color blind person to provide a corresponding color-modified pixel; (f) generate a color-modified image composed of the color-modified pixels and original pixels, the original pixels corresponding to pixels having the input R, G, and B values located outside the filter area in the CIE color space; and (g) cause the electronic display to display the color-modified image.

Embodiments may include one or more of the following features. The data processing apparatus includes a processor and can be programmed to perform step (e) using the processor.

The data processing apparatus includes a field programmable gate array (FPGA) programmed to perform step (e). The system can be a mobile device. The display can be an organic light emitting diode (OLED) display, a liquid crystal display, or a light emitting diode (LED) display.

Among other advantages, active color correction of display images can allow color blind users to experience increased color contrast, fidelity, and detail in device displays thereby improving user satisfaction. The technology can simultaneously support filtration and color correction for multiple types of color blindness allowing color-correction for a number of color blindness types in a single application.

Static filter regions can be stored locally to reduce processing power required for filtration, or actively defined by users or applications to improve color accuracy within a defined color space. Additionally, easily recognizable image color ranges, such as skin tones, can be excluded from filtration to improve user viewing comfort of the color-corrected images.

The calculations necessary for pixel transformation, flagging, and color conversion can be implemented using software loaded onto a device (e.g., a mobile device), integrated device hardware, or purpose-built hardware (e.g., such as an ASIC, or FPGA) allowing for multiple implementation modes. Multiple methods of defining filtration regions are described which utilize varying levels of processing power, adding implementation flexibility based upon at least the processing power of the device or integrated components. In some embodiments, pixel flagging and color transformations of flagged pixels can be done with computational efficiency, allowing for real-time color transformations to be performed (e.g., of streaming videos)

Other advantages will be apparent from the description, the drawings, and the claims.

### BRIEF DESCRIPTION OF THE DRAWINGS

FIG. 1A is a flow chart depicting a color modification process for color blindness remediation.

FIG. 1B is a CIE color space diagram with overlaid color confusion lines and filter area.

FIG. 2 is a flow chart depicting inputting color blindness information of a user.

FIGS. 3A-3C are CIE color space diagrams with overlaid color confusion lines for protanopic, deuteranopic, and tritanopic vision, respectively.

FIG. 4 is a CIE color space diagram with an overlaid RGB color gamut including approximate color regions.

FIG. 5 is a chart including a filter area as defined within a RGB color gamut.

FIGS. 6A-6D are graphs depicting color-adjusting translations away from confusion lines to a new area of a RGB color gamut.

FIG. 7 is a flow chart depicting an alternative color modification process for color blindness remediation.

FIGS. 8A-8F are bar charts depicting the redistribution of RGB color values to create color-modified pixels.

FIG. 9 is a schematic diagram of computing systems for displaying color-modified images to a viewer.

In the figures, like symbols indicate like elements.

### DETAILED DESCRIPTION

Disclosed herein is a technology for correcting a portion of image color information within a display's color gamut to reduce color confusion in certain viewers, e.g., viewers that experience a form color blindness (color blind persons).

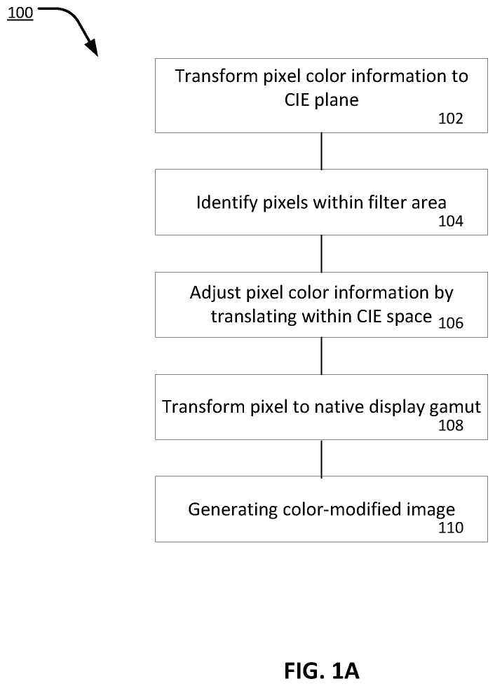

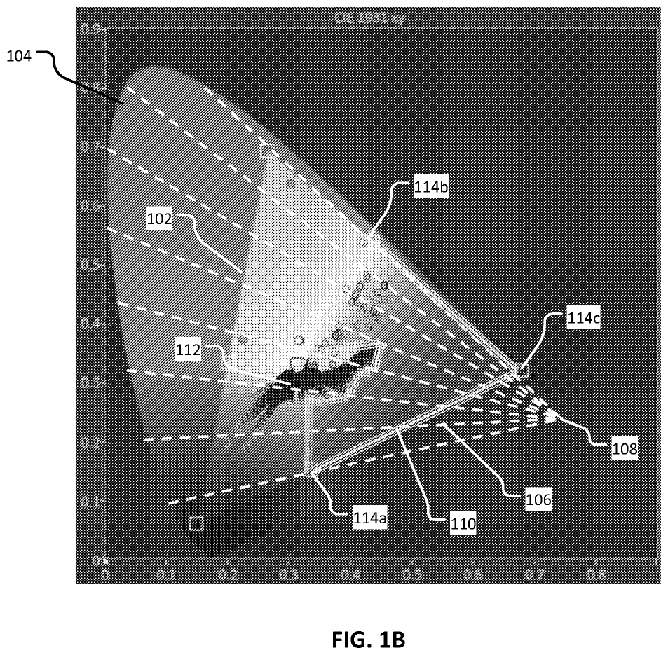

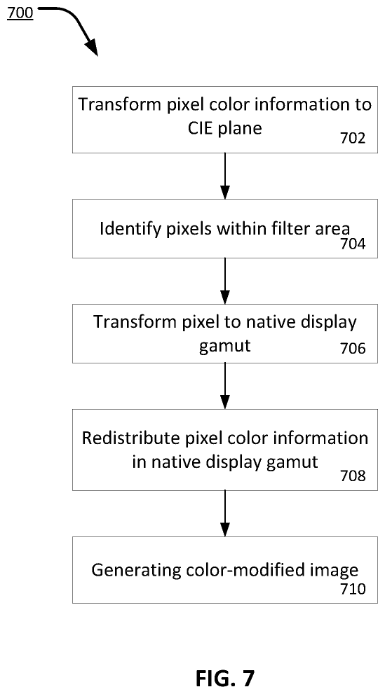

Referring to FIG. 1A, steps of a method for color blindness remediation for a display are shown in a flow chart 100. The method modifies the color of received images based upon user color blindness information received from a user and based on the color gamut of the display. The images can include still images or dynamic images, such as digital video or computer generated dynamic images, e.g., images generated in a video game. An additional color blindness remediation method is described further herein with reference to FIG. 7, below.

Digital images are typically composed of an array of pixels, each having three values corresponding to the intensity level of the sub-pixels for that pixel. For example, the color of many digital image pixels are defined by RGB or CYM values.

The system transforms the color information of the array of pixels composing an image from the native color gamut to corresponding x and y coordinates within the CIE color space (102). The method calculates CIE xy color coordinates for each pixel based upon the native color gamut pixel color information.

The following is an example transformation from the RGB to the xy-plane (i.e. 1931 CIE color space) using the Adobe RGB (1998) transformation matrix. First, the RGB values are used to calculate X, Y, Z tristimulus values by the following transformation:

$$( \begin{matrix}X \\ Y \\ Z\end{matrix} ) = ( \begin{matrix}0.5767 & 0.1855 & 0.1881 \\ 0.2973 & 0.6273 & 0.0752 \\ 0.027 & 0.0706 & 0.9911\end{matrix} ) ( \begin{matrix}R \\ G \\ B\end{matrix} )$$

where R, G, and B are values representing the red, green, and blue sub-pixel intensity value, respectively. X, Y, and Z are tristimulus values representing the 1931 CIE color space encompassing all colors that are perceivable to a person with average eyesight. The chromaticity color coordinate is then specified by two derived parameters x and y, two of the three normalized values being functions of all three tristimulus values X, Y, and Z:

$$x = \frac{X}{X + Y + Z} , y = \frac{Y}{X + Y + Z} , z = \frac{Z}{X + Y + Z}$$

$$and$$

$$x + y + z = 1$$

The 1931 CIE xy color coordinates (x, y) (e.g., chromaticity coordinates) determine the color of the pixel in CIE space. In general, other transformation matrices can be chosen based upon color space, calculation efficiency, or other functional requirements.

After transforming the pixel's RGB values to xy coordinates, the method identifies pixels including 1931 CIE xy color coordinates within a pre-established filter area (104) corresponding to a region of the CIE color space where a color blind viewer will experience reduced color perception, and flags the identified pixels. The method adjusts flagged pixel color information by translating (106) flagged pixel color coordinates (e.g., (x₁, y₁)) within the CIE color space to a second color coordinate (e.g., (x₂, y₂)) away from a confusion line. An adjustment vector is determined based upon one or more criteria such as distance to a gamut feature, distance to a filter area feature, or distance to a confusion line. The flagged color coordinate is adjusted a distance along the adjustment vector, thereby increasing absolute distance from a confusion line by adjusting the color coordinate toward a region of lower color confusion.

Adjusted pixel color coordinates (108) are transformed into the native display color space. The following is an example transformation from translated 1931 CIE color space coordinates (x₂, y₂) to RGB (e.g., (R₂, G₂, B₂)) using the Adobe RGB (1998) transformation matrix:

$${Y}_{2} = {Y}_{1}$$

$${X}_{2} = \frac{{x}_{2} {Y}_{2}}{{y}_{2}}$$

$${Z}_{2} = \frac{( 1 - {x}_{2} - {y}_{2} ) {Y}_{2}}{{y}_{2}}$$

$$( \begin{matrix}{R}_{2} \\ {G}_{2} \\ {B}_{2}\end{matrix} ) = ( \begin{matrix}2.0413 & - 0.5649 & - 0.3446 \\ - 0.9692 & 1.876 & 0.0415 \\ 0.0134 & - 0.1183 & 1.0154\end{matrix} ) ( \begin{matrix}{X}_{2} \\ {Y}_{2} \\ {Z}_{2}\end{matrix} )$$

Pixels including adjusted color coordinates (e.g., color-modified pixels) are combined with un-adjusted pixels to form a color-modified image (110) for display to the user. The color-modified image includes pixels with color information more easily perceived by the color blind user.

FIG. 1B shows a color gamut 102 of a display device in the 1931 CIE chromaticity diagram 104. The color gamut 102 is shown as a triangle defined by vertices 114a, 114b, and 114c, which correspond to the x-y color coordinates of the blue, green, and red sub-pixels of the display. Accordingly, while the diagram 104 depicts the chromaticities available to the human eye, the color gamut 102 represents the subset of chromaticities that the display can generate. In other words, images displayed on a device are composed of an array of pixels, each pixel including subpixels representing color information falling within the device's color gamut.

The orthogonal color values of a display gamut transform into the CIE color space 104 thereby defining the color gamut 102 vertices. Edges connecting the vertices define the color gamut 102 boundaries. The display is capable of producing any color within the triangular color gamut 102 area.

Shown overlaid the chromaticity diagram 104 are dashed color confusion lines 106 radiating from a copunctual point 108. The copunctual point 108 shown in FIG. 1B is the protanopic copunctual point 108. Color blind users experience difficulty perceiving color differences between color coordinates along a color confusion line, causing color confusion between connected color coordinates.

To remediate color confusion in a color blind user, the method adjusts pixel color information from original CIE color coordinates away from the confusion line 106 connecting the original color coordinate to copunctual point 108 to a new color coordinate more easily perceived by the user. The method defines a filter area 110 in the gamut 102 corresponding with user color blindness information, e.g., protanopia, deuteranopia, or tritanopia, and pixels with color coordinates falling within the filter area are adjusted. Color gamut edges define boundaries of the filter area 110 as well as additional boundaries defined by the user or method. For example, in FIG. 1B color coordinates falling within filter area 110 are adjusted away from confusion lines 106.

Filter area 110 outlined in FIG. 1B is based upon a protanopic user lacking red-sensing cone cells. Such a user generally cannot distinguish pixels with red hues from pixels with green hues. The method transforms color coordinates falling within the filter area 110 into areas the user is more capable of perceiving. Alternative filter areas 110 can be defined by the system or by the user adjusting for the color blindness information to be corrected.

Additionally, filter area 110 is shaped to exclude certain color coordinates 112 corresponding to specific tones to remain unmodified. For example, the group of color coordinates 112 shown correspond to human skin tones which may cause added confusion in non-color blind and color blind users if adjusted. More generally, filter areas can be shaped to exclude or include other color coordinates as desired.

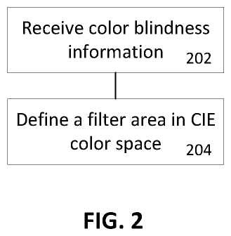

The method uses as input, user color blindness information such as color blindness type or degree, to define the filter area such as filter area 110. Referring now to FIG. 2, steps for determining the color blindness information of a user are shown in flow chart 200. In initial step 202, the user inputs information about their color blindness into the system. For example, this can be performed where the user inputs information into a user device, such as a smart-phone, laptop, tablet computer, PC computer or other device that includes a display. The interface can be any suitable interface type, such as an app, web portal, physical switch, touchscreen, etc. In some implementations, the color blindness information can be supplied to a networked server system which provides the image processing necessary to perform the method.

The information provided by the user can include the type of color blindness (e.g., protanopia, deuteranopia, or tritanopia), degree of color blindness, and/or a correction scale. The color blindness information can be input using any external or integrated input device available to the user device such as a touchscreen, keyboard, or microphone. In some implementations, the user device receives color blindness information stored in a local memory or storage device corresponding with a user identifier such as a screen name, login, or password.

In some embodiments, the system can prompt the user for information about their color perception, e.g., by presenting images of different colors to the user and prompting them to identify which color combinations are easier to discern.

Responsive to the input user color blindness information, the system defines a filter area in a CIE color space (step 204). In general, the filter area is a subset of the display's color gamut and represents those colors which should be adjusted in an image to remediate the viewer's color blindness. As such, the filter area depends on the information received in step 202 about the user's color blindness. For example, a protanopic user experiences color confusion between red and green hues, e.g., red-green color blind, along the color confusion lines 106 of FIG. 1B due to a lack of red-sensing retinal receptors.

Generally, the filter area is defined by one or more closed polygons in the CIE color space, as discussed more below. In some implementations, the system defines the filter area. For example, the system holds in memory one or more filter areas corresponding to the input viewer color blindness. In some implementations, the system modifies the filter area responsive to the type of image to be viewed (e.g., to accommodate images including human skin tones). The system can be implemented on variety of devices and as such, in some implementations, the system defines a filter area based on the device display color gamut. In some implementations, the user defines or modifies the filter area through interaction with the device.

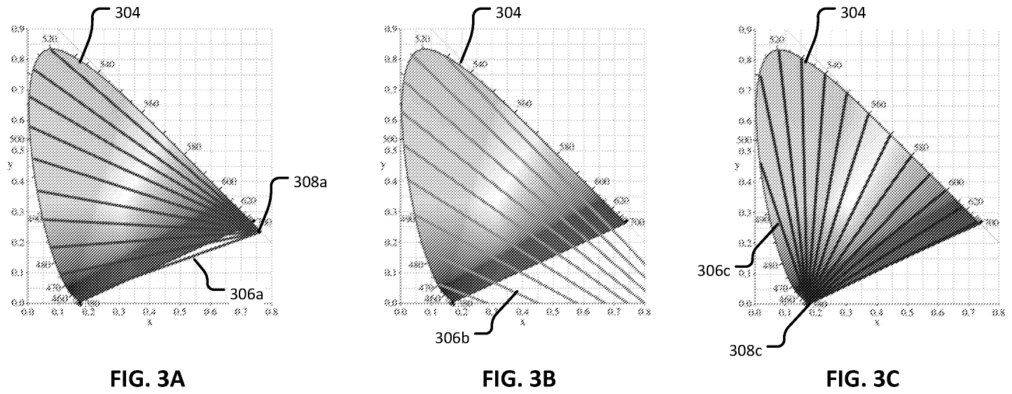

The confusion lines 106 of FIG. 1B correspond with protanopic users (e.g., a protanope). Other color blindness types correspond with unique sets of confusion lines originating from distinct copunctual points for deuteranopia or tritanopic users (e.g., deuteranopes, and tritanopes, respectively). Color confusion is explained in greater detail with reference to FIGS. 3A to 3C showing copunctual points and confusion lines for protanopic, deuteranopic, and tritanopic vision, respectively.

In general, confusion lines are defined between any xy coordinate in the CIE color space and a copunctual point, such as protanopic copunctual point 308a. As such, the confusion lines of FIGS. 3A to 3C are exemplary and not intended to represent complete set of all possible confusion lines.

Color blind users generally cannot perceive color differences between color coordinates along a confusion line. In other words, to take the example of a protanope, a green-hued pixel and a red-hued pixel that lie along a common confusion line will be perceived by the user to have the same color. As the distance between the copunctual point and the perceived color coordinate decreases, hues at that color coordinate are perceived with reduced saturation.

FIG. 3A shows a 1931 CIE chromaticity diagram with protanopic confusion lines 306a radiating from protanopic copunctual point 308a. FIG. 3B shows a 1931 CIE chromaticity diagram with deuteranopic confusion lines 306b radiating from the deuteranopic copunctual point. The deuteranopic copunctual point coordinates are out of the xy coordinate range of FIG. 3B and therefore the copunctual point is not shown. FIG. 3C shows a 1931 CIE chromaticity diagram with tritanopic confusion lines 306c radiating from tritanopic copunctual point 308c.

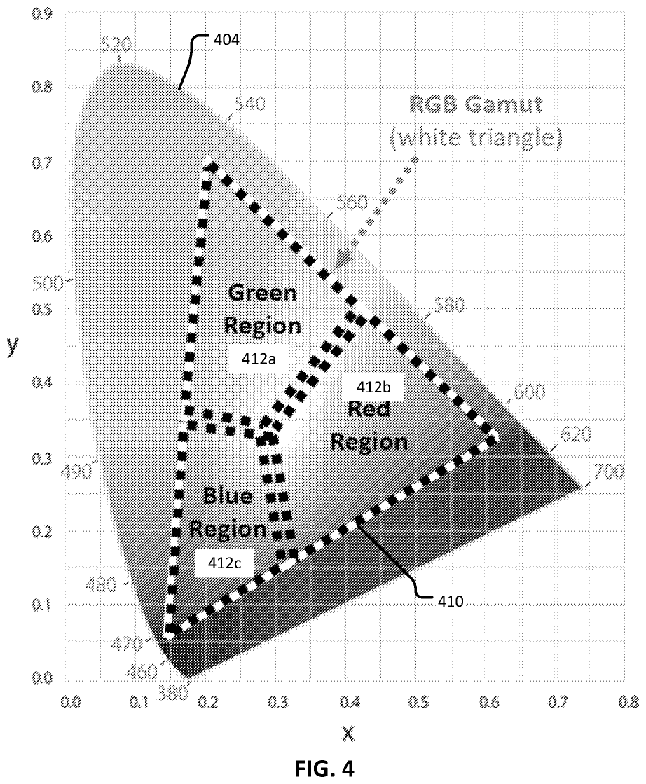

The filter area is determined based upon user color blindness information input to the system (e.g., as described above with reference to FIG. 2), for example, color-adjusting images based upon a protanopic user includes defining a filter area including substantially red hues. FIG. 4 is a 1931 CIE chromaticity diagram 404 with device color gamut 410 divided into three example hue regions 412a-412c. Deuteranopic users experience decreased hue perception of colors within green region 412a, protanopic users experience decreased hue perception in red region 412b, and tritanopic users experience decreased hue perception in blue region 412c. The device uses color blindness information of the user to define filter areas substantially including the color regions of poorly perceived hues.

The filter area is defined by the system as a sequence of connected linear segments, each linear segment defining one border of the filter area. In general, at least three linear segments define a closed polygon, e.g., a triangle, though more define higher order polygons. In some implementations, the user defines the one or more linear segments or polygon of the color gamut filter areas.

In certain implementations, the system creates a list of n linear, non-intersecting segments by defining first and second coordinates within the display color gamut, fᵢ=([x₀, y₀], [x_{f}, y_{f}]). The second coordinates of a first linear segment and the first coordinates of the adjoining segment are equal, and the first coordinates of the first segment, f₁−[x₀, y₀]₁, and the second coordinate of the last segment fₙ=[x_{f}, y_{f}]ₙ and equal, e.g., [x₀, y₀]₁=[x_{f}, y_{f}]ₙ, thereby defining a closed polygonal filter area. The system calculates numerical approximations of the lines between the first and second coordinates for each linear segment and stores the approximations in memory.

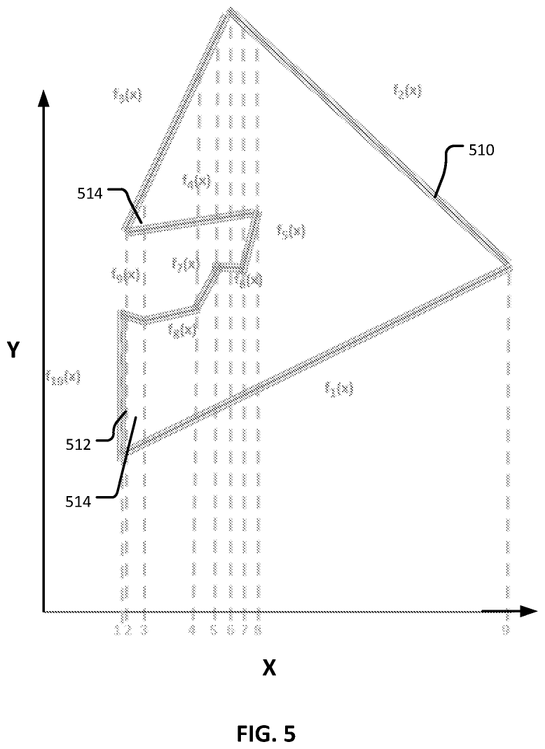

FIG. 5 is a first example of a closed polygon including ten linear segments, f₁ through f₁₀. The ten linear segments define filter area 502. The system flags an image pixel for adjustment if the pixel color coordinates, e.g., [pixel_x, pixel_y] fall within the filter area. An array of logical AND, OR, and inequality statements determine whether the pixel color coordinate is within the filter area. The system flags pixels with color coordinates within the filter area for adjustment.

The system divides the filter area into sub-divisions based upon the first and second x coordinate of the set of linear segments. The filter area 510 of FIG. 5 includes eight sub-divisions numerated from lowest x coordinate to largest, e.g., progressing to the right on the x axis of FIG. 5. The first and second x coordinates of f₁₀(x) are equal, thereby defining f₁₀(x) as a vertical line connecting coordinates of f₉ and f₁. The first sub-division x range begins with the lowest x coordinate, e.g., f₁₀(x), f₉(x), and f₁(x) with further sub-divisions having higher x coordinates. As an example, the first sub-division can include the x coordinate range from 0.3 to 0.39, the second sub-division includes the x coordinate range from 0.4 to 0.49, etc.

The system determines if a pixel color coordinate is within a sub-division by comparing the pixel x coordinate to the sub-division ranges. If the pixel x coordinate is not within one of the sub-division ranges, the pixel is not flagged for adjustment. If the pixel x coordinate is within one of the sub-division ranges, the system compares the pixel y coordinate to the numerical approximations of the linear segments evaluated at the x coordinate using an algorithm.

Logical statements determine whether a pixel is within a sub-division (e.g., region). For example, region 1 (512) is the polygonal area of FIG. 5 bounded on the left by the dashed line labeled 1 on the x-axis, on the right by the dashed line labeled 2, on the bottom by f₁(x), and on the top by f₉(x). As a second example, region 2 (514) is the combined polygonal areas of FIG. 5 bounded on the left by the dashed line labeled 2 on the x-axis, on the right by the dashed line labeled 3, between f₁(x) and f₉(x), and between f₃(x) and f₄(x). Logical statements describing these regions correspond to:

```
  if pixel_x in region 1:
    if pixel_y > f1 (x) AND < f9(x)
    then flag
  elseif pixel_x in region 2:
    if (pixel_y > f1 (x) AND < f9(x)) OR (pixel y > f4(x) AND <
f3(x)) then flag
```

with further logical statements defining each sub-division stored in the system memory. If the pixel color y coordinate is determined to fall within the y-axis boundaries of the sub-division, the pixel is flagged for adjustment.

The filter area can be defined by at least one bounding segments, wherein edges of the display color gamut provide additional boundaries. Though, in general, the number of segments is limited by the computational capacity of the system.

In some implementations, the boundaries can be determined based upon the pixel y color coordinate rather than the x coordinate. In such cases, the system computes the logical statements comparing the pixel y coordinate to sub-division ranges and flags the pixel for adjustment if pixel x coordinate is within one of the sub-division ranges.

Users experiencing color blindness have difficulty perceiving color information within a region of the CIE color space, as described above. To decrease user color confusion, the system adjusts flagged pixels (e.g., pixels within the defined filter area) away from confusion lines on which flagged pixels fall.

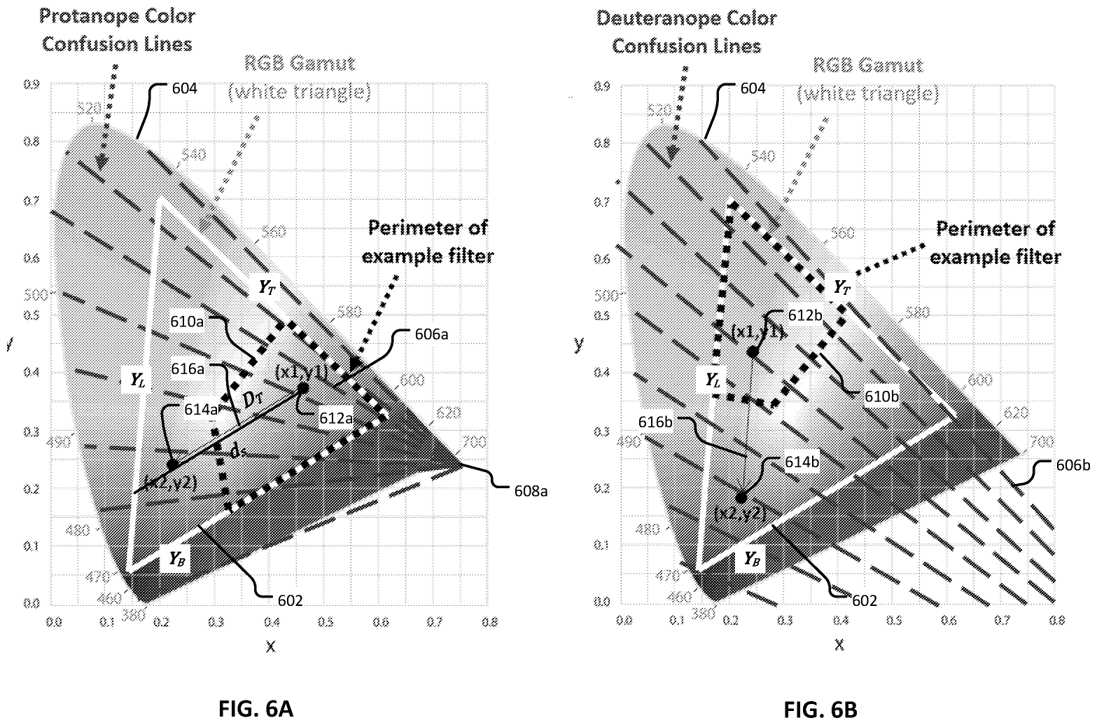

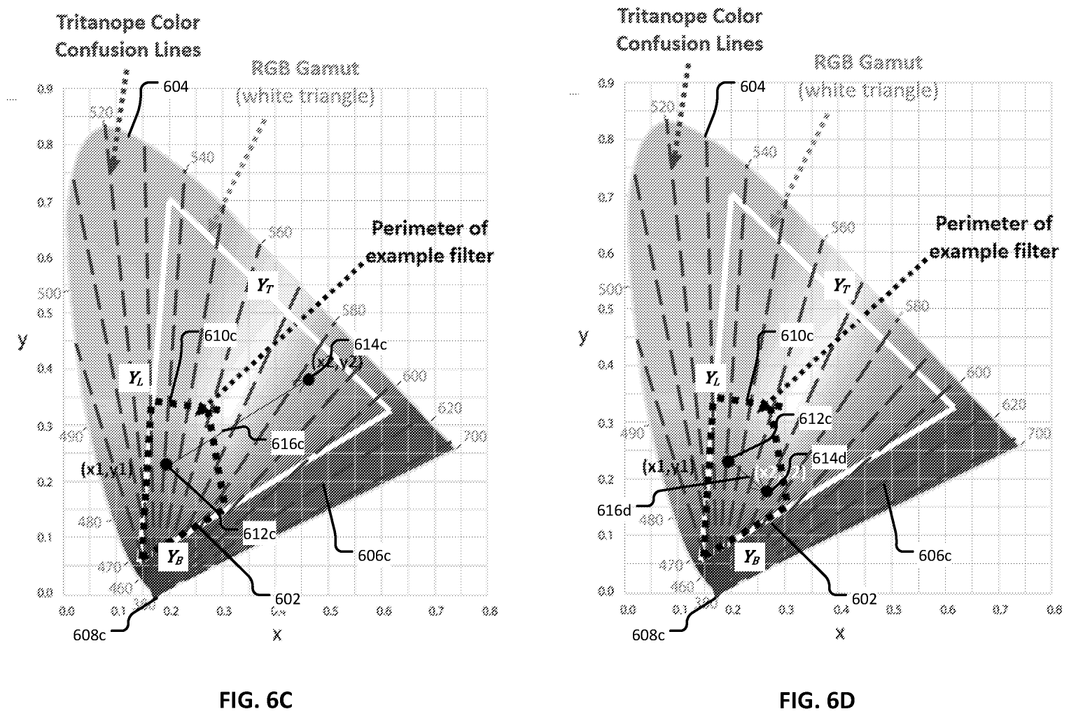

FIGS. 6A-6D are CIE diagrams 604 with display color gamuts 602 and filter areas 610 and flagged pixels before and after adjustment. FIG. 6A includes protanopic confusion lines 606a originating from copunctual point 608a and protanopic filter area 610a, similar to red region 412b. The system determines that a pixel containing color information corresponding to color coordinate 612a (x₁, y₁) falls within filter area 610a and flags the pixel color coordinate 612a for adjustment.

Flagged pixel color coordinates 612a are translated away from confusion lines 606a traversing the translation vector 616a. Translating color coordinate 612a away from confusion line 606a connecting color coordinate 612a and copunctual point 608a constitutes selecting a translation vector 616a and translating the color coordinate 612a along the translation vector 616a by a distance. In general, the translation vector 616a can be selected such that the angle, θ, between the confusion line 606a and translation vector 616a is greater than zero (e.g., θ>0) such that translating the pixel color coordinate 612a a distance along the translation vector 616a increases the color space distance between the color coordinate 612a and the confusion line 606a.

In some implementations, the system selects translation line 616 as a line parallel with a side of the color gamut 602. The sides of color gamut 602 are shown as Y_{T}, Y_{B}, and Y_{L}. Side Y_{T} is the color gamut edge connecting the green and red subpixels, Y_{B} is the color gamut edge connecting the red and blue subpixels, and Y_{L} is the color gamut edge connecting the blue and green subpixels of the display color gamut.

The system selects the side based upon the user color blindness information. Using the protanopic example of FIG. 6A, side Y_{T} is substantially parallel with confusion lines 606a originating from copunctual point 608a. Translation along vector 616a parallel with Y_{T} results in a lower perceived color difference between the original and color-modified color information for a protanopic user whereas translating along vector 616a parallel with Y_{B} results a higher perceived color difference.

The sides of triangular color gamut 602 are lines connecting the primary color coordinates defined by a slope and a y intercept. The slope of translation vector 616a is equal to the slope of the red-blue side of color gamut 602, e.g., the side connecting the red and blue primary color coordinates.

The vector 616a intersects the side of the color gamut 602 at a distance, dₛ, from the color coordinate 612a. Therefore, the distance the color coordinate 612a translates, D_{T}, is between 0 and dₛ, e.g., 0<D_{T}<dₛ. The system includes a scale factor, κ, where 0<κ<1, such that D_{T}=κdₛ and color coordinate 612a translates a fractional amount of the total distance to the color gamut 602 side. For example, scale factor κ=0.60 would translate color coordinate 612a 60% of the distance dₛ.

The system calculates the translated color-modified coordinate according to the following example utilizing the geometric equations (e.g., geometric formulas) described below. The equations describing color gamut 602 of FIG. 6A are:

Y_{L}(x)=9.36*x−1.297

Y_{T}(x)=−0.92*x+0.912

Y_{B}(x)=0.54*x−0.018

The equation for translation vector 616a with slope equaling the slope of Y_{B}(e.g., 0.54) is then

Yₚ(x)=0.54*x+bₚ

where bₚ=y₁−0.54*x₁ and bₚ is the y-axis intercept coordinate for Yₚ. The system calculates the x coordinate at which Yₚ and Y_{L} intersect (Yₚ(x) Y_{L}(x)), e.g.,

$$( {x}^{*} = \frac{( {y}_{1} - 0.54 {x}_{1} ) + 1.297}{8.852} )$$

corresponding to the maximum color-modified x coordinate.

The system then calculates new color coordinates 614a (x₂, y₂) using the difference between x₁ and x*, scaled to a fractional amount by K using the following equations:

x₂=x₁−κ(x₁−x*)

y₂=0.54x₂+bₚ

The system updates the color coordinates for the flagged pixel to the color-modified color coordinates before transforming the pixel color information back to the original color space, thereby generating a color-modified pixel in the original color space.

FIG. 6B is a CIE diagrams 604 with display color gamut 602 and deuteranopic filter areas 610b. The system translates the pixel color coordinate 612b from the filter area 610b along translation vector 616b parallel with side Y_{L}. The system performs similar calculations as described above but to translate color coordinate 612b along vector 616b to color-modified coordinate 614b.

FIGS. 6C and 6D are CIE diagrams 604 with display color gamuts 602 and pixel color coordinates 612c within tritanopic filter areas 610c. FIG. 6C depicts a first translation wherein the system translates the color coordinate 612c along translation vector 616c parallel with side Y_{B}, parallel with the translation shown in FIG. 6A.

An alternative adjustment is shown in FIG. 6D wherein the system adjusts pixel color coordinates 612c along adjustment vector 616d parallel with side Y_{T}. Confusion lines 606c radiating from copunctual point 608c form larger angles with side Y_{T} with respect to the angles formed with intersecting side Y_{B}. Adjusting color coordinates along a vector 616d parallel with Y_{T} results in color-modified color coordinates 614d being adjusted at a greater angle to confusion lines 606c.

While the color transformation of flagged pixels described above involves adjusting the color of each flagged pixel within the two-dimensional 1931 CIE color space and then transforming the new x, y values to the display's native pixel color format, other implementations are possible.

For example, alternatively, flagged pixels can be color-modified in their native space, e.g., sRGB. For example, the system redistributes the relative weights, e.g., the specific color value intensity with respect to the normalized sum of all color values, of flagged pixel color information within the sRGB space. The color transformation of a pixel in native format can involve reducing the intensity of one sub-pixel color value while increasing (e.g., proportionally) another. The reduced and increased color values typically correspond with at least the color blindness form.

The alternative adjustment method is described with reference to FIG. 7, in which steps of a method for color blindness remediation for a display are shown in a flow chart 700. The system transforms the color information of the array of pixels composing an image from the native color gamut to corresponding x and y coordinates within the CIE color space (702), similar to step (102) above. The method identifies pixels including CIE color coordinates within the filter area (704) and flags the identified pixels, similar to step (104) above. The system then includes an array of flagged and unflagged pixels.

The method transforms the array of flagged and unflagged pixels into the native display color space (706), such as RGB. The method the redistributes sub-pixel intensity values (708) to adjust the perceived color away from confusion lines. Redistribution includes reducing a sub-pixel intensity value by an amount and increasing a sub-pixel intensity value corresponding to the color blindness type being corrected.

Pixels including adjusted color coordinates are combined with un-adjusted pixels to form a color-modified image (710) for display to the user, similar to step (110) above.

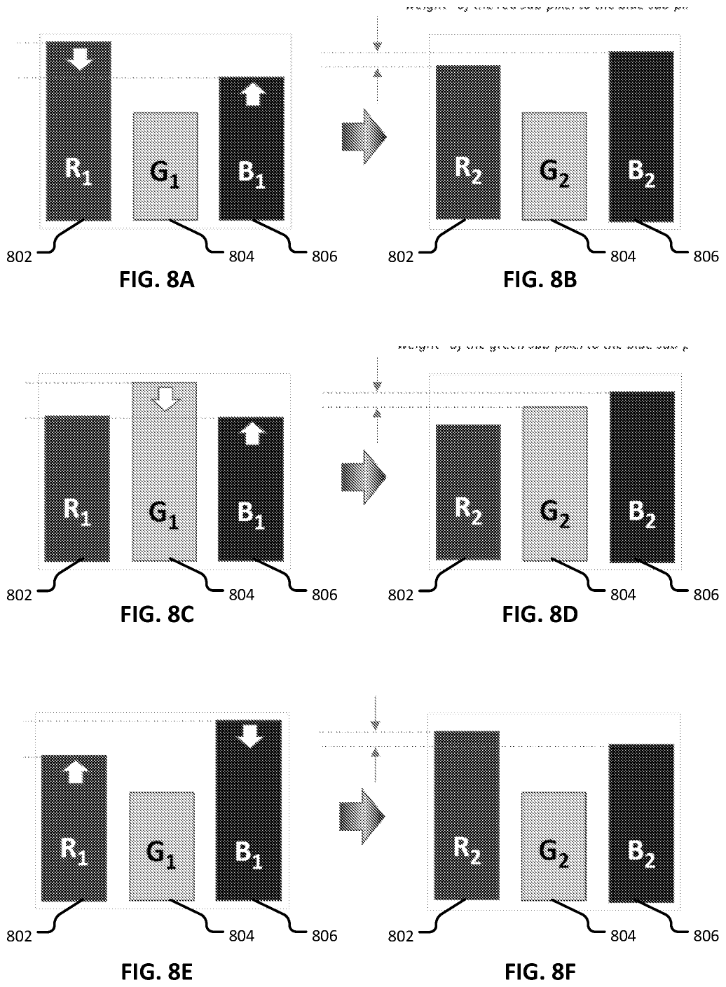

FIGS. 8A-8F are example bar charts showing relative weights (e.g., intensities) of pixel color values including red color values, green color values, and blue color values, with larger bar scale representing higher color values. The system redistributes a portion of a relative weight of at least one of the input red, green, or blue (e.g., R, G, B) subpixels for each flagged pixel within in the filter area. For example, correcting a pixel flagged for modification for protanopic vision can include reducing the intensity of the red color value while increasing the blue color value which are more readily perceived by the user.

Sub-pixel intensity value redistribution is discussed with reference to FIGS. 8A-8F. FIG. 8A is a pixel color information bar charts depicting the input subpixel color values for one protanopic flagged pixel, including red color value 802, green color value 804, and blue color value 806, e.g., [R₁, G₁, B₁]. Flagged pixels with in a protanopic filter area include larger relative red color value 802 weights than green color value 804 or blue color value 806, as shown in FIG. 8A. Additionally, protanopic color confusion lines originating from the protanopic copunctual point align along red and green color areas of the display color gamut. As such, the system redistributes red color value 802 to the blue color value 806 to adjust the perceived color for protanopic users.

The system determines a color value difference between the R input color value and the B input color value, e.g., R₁−B₁. The system then adds a portion, e.g., a fractional amount, of the red-blue color value difference to the blue color value to determine adjusted color values. The system calculates the portion added to the blue color value using a scalar factor, such as γ, with a value between 0 and 1 (e.g., 0<γ<1). The system selects a γ value based upon at least the color blindness information of the user. For example, scalar factor γ=0.90 would redistributed 90% of the color value difference from the red color value to the blue.

The system calculates new protanopic color-modified color values, e.g., [R₂, G₂, B₂], using the following formulae:

R₂=R₁−γ*(R₁−B₁)

G₂=G₁

B₂=B₁+γ*(R₁−B₁)

Protanopic color-modified pixel color values 802, 804, 806 are shown in FIG. 8B including a higher blue color value 806.

Responsive to deuteranopic user color blindness information, the system adjusts pixel color values by redistributing green color value to the blue color value. Flagged pixels with in a deuteranopic filter area include larger relative green color values 804 than red 802 or blue 806, as shown in FIG. 8C. The system determines a color value difference between the G input color value 804 and the B input color value 806, e.g., G₁−B₁. The system then adds a portion of the green-blue color value difference to the blue color value 806 to determine adjusted color values. The system calculates deuteranopic color-modified color values, [R₂, G₂, B₂], using the following formulae:

R₂=R₁

G₂=G₁−γ*(G₁−B₁)

B₂=B₁+γ*(G₁−B₁)

Deuteranopic color-modified pixel color values are shown in FIG. 8D including a higher blue color value 806.

Responsive to tritanopic user color blindness information, the system adjusts pixel color values by redistributing blue color value to the red color value. Flagged pixels with in a tritanopic filter area include larger relative blue color values 806 than red 802 or green 804, as shown in FIG. 8E. The system determines a color value difference between the B input color value 806 and the R input color value 802, e.g., B₁−R₁. The system then adds a portion of the blue-red color value difference to the red color value 802 to determine adjusted color values. The system calculates tritanopic color-modified color values, [R₂, G₂, B₂], using the following formulae:

R₂=R₁+γ*(B₁−R₁)

G₂=G₁

B₂=B₁−γ*(B₁−R₁)

Tritanopic color-modified pixel color values are shown in FIG. 8F including a higher red color value 802.

The color adjusting calculations described above, and depicted in FIGS. 8A-8F, can alternatively be expressed in three equations utilizing twelve coefficients:

R₂=R₁+(δ_{R})*γ*(α_{R}*R₁+β_{R}*B₁+θ_{R}*G₁)

G₂=G₁+(δ_{G})*γ*(α_{G}*R₁+β_{G}*B₁+θ_{G}*G₁)

B₂=B₁+(δ_{B})*γ*(α_{B}*R₁+β_{B}*B₁+θ_{B}*G₁)

where α, β, δ, and θ values are binary integer values between −1 and 1, e.g., −1, 0, or 1, corresponding with the user color blindness information. For protanopic vision, the values are:

$$( \begin{matrix}{δ}_{R} & {α}_{R} & {β}_{R} & {θ}_{R} \\ {δ}_{G} & {α}_{G} & {β}_{G} & {θ}_{G} \\ {δ}_{B} & {α}_{B} & {β}_{B} & {θ}_{B}\end{matrix} ) = ( \begin{matrix}- 1 & 1 & 0 & - 1 \\ 0 & 0 & 0 & 0 \\ 1 & 1 & 0 & - 1\end{matrix} )$$

For deuteranopic vision, the values are:

$$( \begin{matrix}{δ}_{R} & {α}_{R} & {β}_{R} & {θ}_{R} \\ {δ}_{G} & {α}_{G} & {β}_{G} & {θ}_{G} \\ {δ}_{B} & {α}_{B} & {β}_{B} & {θ}_{B}\end{matrix} ) = ( \begin{matrix}0 & 0 & 0 & 0 \\ - 1 & 0 & 1 & - 1 \\ 1 & 0 & 1 & - 1\end{matrix} )$$

For tritanopic vision, the values are:

$$( \begin{matrix}{δ}_{R} & {α}_{R} & {β}_{R} & {θ}_{R} \\ {δ}_{G} & {α}_{G} & {β}_{G} & {θ}_{G} \\ {δ}_{B} & {α}_{B} & {β}_{B} & {θ}_{B}\end{matrix} ) = ( \begin{matrix}1 & - 1 & 0 & 1 \\ 0 & 0 & 0 & 0 \\ - 1 & - 1 & 0 & 1\end{matrix} )$$

The system then generates a color-modified image by combining the color-modified pixels with the original, un-flagged, un-modified pixels from the original image. The modified- and un-modified pixels are combined sequentially according to their original coordinate location within the original image to generate the color-modified image. The system displays the generated color-modified image to the user.

While certain implementations have been described, other implementations are possible. For example, while the foregoing examples are described with reference to the 1931 CIE color space, other two-dimensional color spaces can be used (e.g., 1964 CIE color space or the 1976 CIE color space).

The calculations described herein can be performed with a system using one or more processors integrated with the components of the system. Alternatively, the calculations can be performed using one or more graphics processing unit (GPU). A GPU is a dedicated graphics rendering device used to generate computerized graphics for display on a display. GPUs are built with a highly-parallel structure that provides more efficient processing than typical, general purpose central processing units (CPUs) for a range of complex algorithms. For example, the complex algorithms may correspond to representations of three-dimensional computerized graphics. In such a case, a GPU can implement a number of primitive graphics operations to create three-dimensional images for display on a display more quickly than using a CPU to draw the image for display on the display.

In some implementations, an integrated circuit device designed to perform the calculations described herein, such as a field-programmable gate array (FPGA) or an application-specific integrated circuit (ASIC), calculates the color transformations and adjustments. The integrated circuit device communicates with other components of the system, for example wired or wireless communication, and receives the image pixel information, performs the calculations necessary to generate a color-modified image, and transmits the color-modified image to a device for display.

In some implementations, the image pixel information is modified based on user color blindness information by a networked computing system (e.g., video streaming service) before the color-modified image is transmitted to the user device through wired or wireless communications networks.

Displays with higher dimensional gamut color spaces (i.e., with pixels composed of more than three primary colors), such as hexachrome or CMYK, produce non-triangular shapes in the CIE color space, such as color space 104. However, the gamut remains bounded by edges connecting transformed coordinate vertices. In this manner, the method disclosed herein can be used to adjust image color information in any gamut color space.

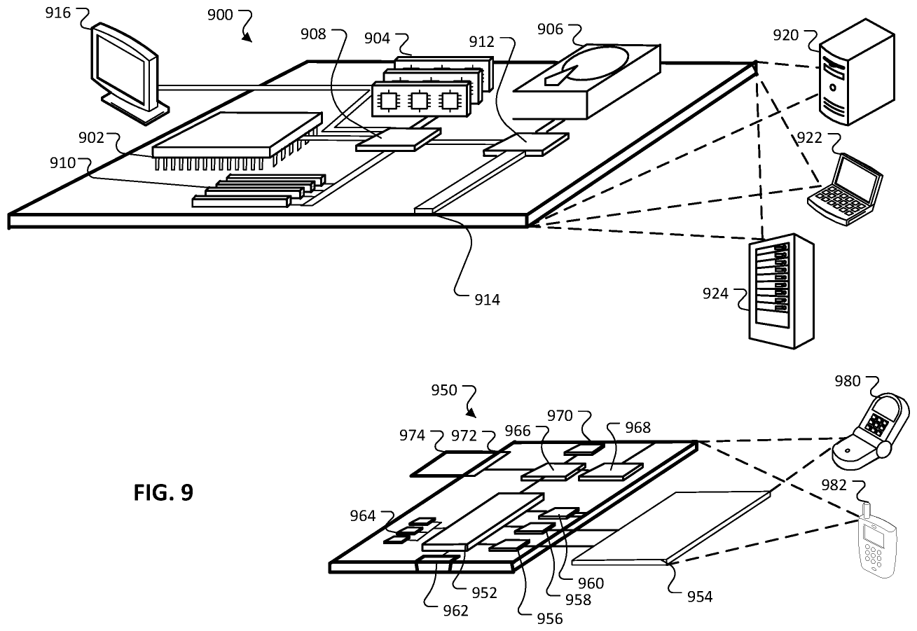

As noted previously, the systems and methods disclosed above utilize data processing apparatus to implement aspects of generating a color-modified image for display to a viewer experiencing color blindness as described herein. FIG. 9 shows an example of a computing device 900 and a mobile computing device 950 that can be used as data processing apparatuses to implement the techniques described. The computing device 900 is intended to represent various forms of digital computers, such as laptops, desktops, workstations, personal digital assistants, servers, blade servers, mainframes, and other appropriate computers. The mobile computing device 950 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 900 includes a processor 902, a memory 904, a storage device 906, a high-speed interface 908 connecting to the memory 904 and multiple high-speed expansion ports 910, and a low-speed interface 912 connecting to a low-speed expansion port 914 and the storage device 906. Each of the processor 902, the memory 904, the storage device 906, the high-speed interface 908, the high-speed expansion ports 910, and the low-speed interface 912, are interconnected using various busses, and may be mounted on a common motherboard or in other manners as appropriate. The processor 902 can process instructions for execution within the computing device 900, including instructions stored in the memory 904 or on the storage device 906 to display graphical information for a GUI on an external input/output device, such as a display 916 coupled to the high-speed interface 908. In other implementations, multiple processors and/or multiple buses may be used, as appropriate, along with multiple memories and types of memory.

The memory 904 stores information within the computing device 900. In some implementations, the memory 904 is a volatile memory unit or units. In some implementations, the memory 904 is a non-volatile memory unit or units. The memory 904 may also be another form of computer-readable medium, such as a magnetic or optical disk.

The storage device 906 is capable of providing mass storage for the computing device 900. In some implementations, the storage device 906 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 902), perform one or more methods, such as those described above. The instructions can also be stored by one or more storage devices such as computer- or machine-readable mediums (for example, the memory 904, the storage device 906, or memory on the processor 902).

The high-speed interface 908 manages bandwidth-intensive operations for the computing device 900, while the low-speed interface 912 manages lower bandwidth-intensive operations. Such allocation of functions is an example only. In some implementations, the high-speed interface 908 is coupled to the memory 904, the display 916 (e.g., through a graphics processor or accelerator), and to the high-speed expansion ports 910, which may accept various expansion cards (not shown). In the implementation, the low-speed interface 912 is coupled to the storage device 906 and the low-speed expansion port 914. The low-speed expansion port 914, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 900 may be implemented in a number of different forms, as shown in the figure. For example, it may be implemented as a standard server 920, or multiple times in a group of such servers. In addition, it may be implemented in a personal computer such as a laptop computer 922. It may also be implemented as part of a rack server system 924. Alternatively, components from the computing device 900 may be combined with other components in a mobile device (not shown), such as a mobile computing device 950. Each of such devices may contain one or more of the computing device 900 and the mobile computing device 950, and an entire system may be made up of multiple computing devices communicating with each other.

The mobile computing device 950 includes a processor 952, a memory 964, an input/output device such as a display 954, a communication interface 966, and a transceiver 968, among other components. The mobile computing device 950 may also be provided with a storage device, such as a micro-drive or other device, to provide additional storage. Each of the processor 952, the memory 964, the display 954, the communication interface 966, and the transceiver 968, are interconnected using various buses, and several of the components may be mounted on a common motherboard or in other manners as appropriate.

The processor 952 can execute instructions within the mobile computing device 950, including instructions stored in the memory 964. The processor 952 may be implemented as a chipset of chips that include separate and multiple analog and digital processors. The processor 952 may provide, for example, for coordination of the other components of the mobile computing device 950, such as control of user interfaces, applications run by the mobile computing device 950, and wireless communication by the mobile computing device 950.

The processor 952 may communicate with a user through a control interface 958 and a display interface 956 coupled to the display 954. The display 954 may be, for example, a TFT (Thin-Film-Transistor Liquid Crystal Display) display, a liquid crystal (LCD) display, a light emitting diode (LED) display, or an OLED (Organic Light Emitting Diode) display, or other appropriate display technology. The display interface 956 may comprise appropriate circuitry for driving the display 954 to present graphical and other information to a user. The control interface 958 may receive commands from a user and convert them for submission to the processor 952. In addition, an external interface 962 may provide communication with the processor 952, so as to enable near area communication of the mobile computing device 950 with other devices. The external interface 962 may provide, for example, for wired communication in some implementations, or for wireless communication in other implementations, and multiple interfaces may also be used.

The memory 964 stores information within the mobile computing device 950. The memory 964 can be implemented as one or more of a computer-readable medium or media, a volatile memory unit or units, or a non-volatile memory unit or units. An expansion memory 974 may also be provided and connected to the mobile computing device 950 through an expansion interface 972, which may include, for example, a SIMM (Single In Line Memory Module) card interface. The expansion memory 974 may provide extra storage space for the mobile computing device 950, or may also store applications or other information for the mobile computing device 950. Specifically, the expansion memory 974 may include instructions to carry out or supplement the processes described above, and may include secure information also. Thus, for example, the expansion memory 974 may be provide as a security module for the mobile computing device 950, and may be programmed with instructions that permit secure use of the mobile computing device 950. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier. The instructions, when executed by one or more processing devices (for example, processor 952), perform one or more methods, such as those described above. The instructions can also be stored by one or more storage devices, such as one or more computer- or machine-readable mediums (for example, the memory 964, the expansion memory 974, or memory on the processor 952). In some implementations, the instructions can be received in a propagated signal, for example, over the transceiver 968 or the external interface 962.

The mobile computing device 950 may communicate wirelessly through the communication interface 966, which may include digital signal processing circuitry where necessary. The communication interface 966 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 968 using a radio-frequency. In addition, short-range communication may occur, such as using a Bluetooth, WiFi, or other such transceiver (not shown).

The mobile computing device 950 may be implemented in a number of different forms, as shown in the figure. For example, it may be implemented as a cellular telephone 1480. It may also be implemented as part of a smart-phone 1482, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

Other embodiments are in the following claims.

1. A method, comprising:
(a) receiving information about a color blindness of a viewer of an electronic display;
(b) receiving information about an input image comprising input R, G, and B values for each of a plurality of pixels composing the input image;
(c) transforming each pixel's input R, G, and B values to a corresponding x value and a corresponding y value, the x and y values being in a CIE color space;
(d) identifying which of the pixels are within a filter area in the CIE color space based on the x, y values;
(e) adjusting a color of each of the pixels that are within the filter area away from a confusion line in the CIE space for the color blind person to provide a corresponding color-modified pixel;
(f) generating a color-modified image composed of the color-modified pixels and original pixels, the original pixels corresponding to pixels having the input R, G, and B values located outside the filter area in the CIE color space; and
(g) displaying the color-modified image with the electronic display.

2. The method of claim 1, further comprising defining the filter area in the CIE color space based on the information about the color blindness of the viewer.

3. The method of claim 2, wherein the filter area is defined by specifying one or more lines defining a border of the filter area.

4. The method of claim 3, wherein the one or more lines define a polygon in the CIE color space within a color gamut of the electronic display.

5. The method of claim 1, wherein the color of each pixel within the filter area is adjusted by translating the x, y coordinates of the pixel in the CIE color space according to a geometric formula.

6. The method of claim 5, wherein the electronic display has a color gamut defined by a triangle in the CIE color space and adjusting the color of each pixel that is within the filter area away from the line of color confusion comprises translating the x, y coordinates of the pixel along a line parallel to a side of the triangle defining the color gamut.

7. The method of claim 6, wherein the x, y coordinates for a pixel within the filter area is translated by a fractional amount of a distance of the x, y coordinates to a side of the triangle defining the color gamut.

8. The method of claim 6, wherein the confusion line is for a protanope and the side of the triangle defining the color gamut is the side defined by blue and red sub-pixels of the electronic display.

9. The method of claim 6, wherein the confusion line is for a deuteranope and the side of the triangle defining the color gamut is the side defined by blue and green sub-pixels of the electronic display.

10. The method of claim 6, wherein the confusion line is for a tritanope and the side of the triangle defining the color gamut is the side defined by blue and red sub-pixels of the electronic display.

11. The method of claim 6, wherein the confusion line is for a tritanope and the side of the triangle defining the color gamut is the side defined by green and red sub-pixels of the electronic display.

12. The method of claim 1, wherein x, y values in the CIE color space for one or more of the color-modified pixels lies within the filter area.

13. The method of claim 1, wherein x, y values in the CIE color space for one or more of the color-modified pixels lies outside the filter area.

14. The method of claim 1, wherein the color of each pixel within the filter area is adjusted by redistributing relative weights of the input R, G, B values for each pixel within the filter area.

15. The method of claim 14, wherein the confusion line is for a protanope and redistributing relative weights of the input R, G, B values for each pixel within the filter area comprises increasing a ratio of the R value to the B value.

16. The method of claim 15, wherein the confusion line is for a dueteranope and redistributing relative weights of the input R, G, B values for each pixel within the filter area comprises increasing a ratio of the B value to the G value.

17. The method of claim 15, wherein the confusion line is for a tritanope and redistributing relative weights of the input R, G, B values for each pixel within the filter area comprises increasing a ratio of the R value to the B value.

18. A computer readable medium containing program instructions for displaying a color-modified image with an electronic display, wherein execution of the program instructions by one or more processors of a computer system causes the one or more processors to carry out the steps of:
(a) receiving information about a color blindness of a viewer of an electronic display;
(b) receiving information about an input image comprising input R, G, and B values for each of a plurality of pixels composing the input image;
(c) transforming each pixel's input R, G, and B values to a corresponding x value and a corresponding y value, the x and y values being in a CIE color space;
(d) identifying which of the pixels are within a filter area in the CIE color spaced based on the x, y values;
(e) adjusting a color of each of the pixels that are within the filter area away from a confusion line in the CIE space for the color blind person to provide a corresponding color-modified pixel;
(f) generating a color-modified image composed of the color-modified pixels and original pixels, the original pixels corresponding to pixels having the input R, G, and B values located outside the filter area in the CIE color space; and
(g) displaying the color-modified image with the electronic display.

19. A system, comprising:
an electronic display; and
a data processing apparatus programmed to:
(a) receive information about a color blindness of a viewer of the electronic display;
(b) receive information about an input image comprising input R, G, and B values for each of a plurality of pixels composing the input image;
(c) transform each pixel's input R, G, and B values to a corresponding x value and a corresponding y value, the x and y values being in a CIE color space;
(d) identify which of the pixels are within a filter area in the CIE color spaced based on the x, y values;
(e) adjust a color of each of the pixels that are within the filter area away from a confusion line in the CIE space for the color blind person to provide a corresponding color-modified pixel;
(f) generate a color-modified image composed of the color-modified pixels and original pixels, the original pixels corresponding to pixels having the input R, G, and B values located outside the filter area in the CIE color space; and
(g) cause the electronic display to display the color-modified image.

20. The system of claim 19, wherein the data processing apparatus comprises a processor and is programmed to perform step (e) using the processor.

21-23. (canceled)
